(19) United States
(12) Patent Application Publication
(10) Pub. No.: US 2022/0346651 A1
(43) Pub. Date: Nov. 3, 2022

(54) DISEASED PERSON DISTINGUISHING DEVICE AND DISEASED PERSON DISTINGUISHING SYSTEM

(71) Applicants: NIPPON AVIONICS CO., LTD., Yokohama-shi, Kanagawa (JP); HYOGO COLLEGE OF MEDICINE, Nishinomiya-shi, Hyogo (JP)

(72) Inventors: Shoichi KIMURA, Yokohama-shi (JP); Kota MATSUMOTO, Yokohama-shi (JP); Yasushi UDA, Yokohama-shi (JP); Eijiro TANJI, Yokohama-shi (JP); Masahiro KOSHIBA, Nishinomiya-shi (JP); Hiromi SHIBATA, Kobe-shi (JP); Osamu HORIE, Nishinomiya-shi (JP)

(73) Assignees: NIPPON AVIONICS CO., LTD., Yokohama-shi, Kanagawa (JP); HYOGO COLLEGE OF MEDICINE, Nishinomiya-shi, Hyogo (JP)

(21) Appl. No.: 17/763,506

(22) PCT Filed: Sep. 29, 2020
(86) PCT No.: PCT/JP2020/036787

§ 371 Date: Mar. 24, 2022

(30) Foreign Application Priority Data
Oct. 4, 2019 (JP) 2019-183691

(51) Int. Cl. A61B 5/01; A61B 5/00

(52) U.S. Cl. CPC A61B 5/015; A61B 5/7425

(57) ABSTRACT

A diseased person distinguishing device includes a temperature pattern calculation unit that calculates a temperature pattern in an exposed region of a subject on the basis of temperature data input from a body surface temperature measurement device that measures the body surface temperature of the subject, and a subject distinguishing unit that applies a learned model that has been learned in advance to the calculated temperature pattern to determine whether the subject is a diseased person or a healthy person.

### TECHNICAL FIELD

The present invention relates to a diseased person distinguishing device and a diseased person distinguishing system.

### BACKGROUND ART

Conventionally, in many facilities, the measurement of the body surface temperature of a subject is periodically performed. As the subject, for example, an inpatient or an outpatient in a hospital facility, a passenger in an airport facility, a visitor in a building or the like, or a child in an educational facility is assumed. In recent years, a thermographic device often used to measure the body surface temperature of the subject in a non-contact manner. The thermographic device can display the body surface temperature of the subject with, for example, a thermal image color-coded in red for high temperature and in blue for low temperature in accordance with the intensity of infrared light emitted from the skin of the subject.

When the subject is infected with a virus and develops the disease, the body surface temperature of the subject rises. For this reason, there has been a case where the thermal image of the subject is captured by a thermographic device before the subject enters a facility to distinguish between a healthy person and a diseased person. A predetermined temperature threshold value has been used to distinguish between a diseased person and a healthy person using the thermal image displayed on the thermographic device. For example, a person whose part having a temperature higher than the temperature threshold value appears on the face surface is identified as a diseased person, and a person whose part having a temperature higher than the temperature threshold value does not appear on the face surface is identified as a healthy person.

Various methods have been attempted to distinguish between a diseased person and a healthy person. For example, Patent Literature 1 discloses a technique for acquiring infrared image data with an eye or a forehead region of a subject as a target region and deriving core body temperature information of the subject.

In addition, a method disclosed in Non Patent Literature 1 by the inventors of the present application is known. Non Patent Literature 1 describes a technique for performing face thermographic measurement for healthy volunteers, influenza type A and type B patients, and febrile patients other than influenza patients, comparing the face thermographic images of the healthy group and the febrile patient group, and deriving the correlation of the face surface temperature of the febrile patients with the body surface temperature thereof.

### CITATION LIST

### Patent Literature

Patent Literature 1: JP 2007-516018 A

### Non Patent Literature

Non Patent Literature 1: Hiromi Shibata and seven others, “Assessment of fever for infection control using thermography—facial thermography in patients with influenza—”, Biomedical Thermology, 2015.01, volume 34, No. 2, p. 54-58

### SUMMARY OF INVENTION

### Technical Problem

Meanwhile, even in a case where the technique disclosed in Patent Literature 1 is used, erroneous distinction such as identifying a healthy person having a high body temperature as a diseased person and identifying a diseased person having a low body temperature as a healthy person may occur. Also, a subject actually infected with a virus whose body temperature has not increased since the disease has not been developed may erroneously be identified as a healthy person. Also, since the surface of the face cools when exposed to air outside of the building, even a diseased person who has fever may erroneously be identified as a healthy person. Also, a threshold value used to distinguish between a diseased person and a healthy person needs to be adjusted to an appropriate value in accordance with the environment in which distinction is performed, and in a case where the threshold value is not adjusted, the probability of erroneous distinction increases. In order to adjust the threshold value to an appropriate value, the adjuster needs to have experience, but when the environment changes, the threshold value needs to be readjusted, which has been an operational problem.

Also, in Non Patent Literature 1, there is no such strong correlation between the body surface temperature and the body temperature as to reliably detect a febrile patient, but the inventors have considered that the accuracy rate for distinction will be improved in a case where the pattern distribution of the face temperature characteristic of febrile patients can be added as a distinction criterion. However, at the time of writing the paper, it is generally considered that the entire face of a febrile patient has a fever, and knowledge about the distribution pattern of the face temperature has not been obtained. Also, it has been unclear how to obtain the distribution pattern of the face temperature characteristic of febrile patients, and thus it has been desired to create a distinction criterion capable of improving the accuracy rate for distinction.

The present invention has been made in view of such a situation, and an object thereof is to distinguish between a diseased person and a healthy person.

### Solution to Problem

In order to achieve at least one of the above objects, a diseased person distinguishing device reflecting an aspect of the present invention includes a temperature pattern calculation unit that calculates a temperature pattern from the temperature in an exposed region of a subject on the basis of temperature data input from a body surface temperature measurement device that measures the body surface temperature of the subject, and a subject distinguishing unit that applies a learned model that has been learned in advance to the calculated temperature pattern to determine whether the subject is a diseased person or a healthy person.

Also, a diseased person distinguishing system reflecting an aspect of the present invention includes not only the aforementioned diseased person distinguishing device but also a learning server that includes a temperature pattern learning unit that learns a temperature pattern by means of a machine learning model and generates a learned model.

### Advantageous Effects of Invention

According to the present invention, by applying a learned model to a temperature pattern for each specific site of a diseased person or a healthy person, it is possible to easily and accurately distinguish between a diseased person and a healthy person.

Problems, configurations, and effects other than those described above will be clarified by the following description of embodiments.

### BRIEF DESCRIPTION OF DRAWINGS

FIG. 1 is a block diagram illustrating an internal configuration example of a diseased person distinguishing system according to a first embodiment of the present invention.

FIG. 2 is a block diagram illustrating an internal configuration example of a control unit according to the first embodiment of the present invention.

FIG. 3 is a block diagram illustrating an internal configuration example of a diseased person distinguishing device according to the first embodiment of the present invention.

FIG. 4 is a block diagram illustrating a hardware configuration example of a computing machine according to the first embodiment of the present invention.

FIG. 5 is a diagram illustrating positions of specific sites according to the first embodiment of the present invention.

FIG. 6 is a diagram for explaining how the temperature pattern of the entire face including the specific sites is normalized according to the first embodiment of the present invention. FIG. 6A illustrates graphs in which the body surface temperatures of a plurality of healthy persons and a plurality of diseased persons obtained by photographing by means of a body surface temperature measurement device are averaged for each part. FIG. 6B illustrates graphs illustrating normalized body surface temperatures of the healthy person and the diseased person.

FIG. 7 is a diagram illustrating display examples of normalized thermal images of the faces of a healthy person and a diseased person according to the first embodiment of the present invention. FIG. 7A illustrates an example of a thermal image of the face of a healthy person, and FIG. 7B illustrates an example of a thermal image of the face of a diseased person.

FIG. 8 illustrates an example of a temperature pattern in a thermal image of the face on a horizontal line passing through the eye inner corners according to the first embodiment of the present invention. FIG. 8A illustrates the horizontal line passing through the eye inner corners of the thermal image of the face of a subject and a list illustrating the difference between a healthy person and a diseased person in terms of the normalized temperature value in the specific site. FIG. 8B illustrates the temperature pattern in the thermal images of the faces of the subjects measured along the horizontal line.

FIG. 9 illustrates an example of a temperature pattern in the thermal image of the face on a horizontal line passing through the nose tip according to the first embodiment of the present invention. FIG. 9A illustrates the horizontal line passing through the nose tip of the thermal image of the face of the subject and a list illustrating the difference between a healthy person and a diseased person in terms of the normalized temperature values in the specific sites. FIG. 9B illustrates the temperature pattern in the thermal images of the faces of the subjects measured along the horizontal line.

FIG. 10 is a list illustrating distinction results in the case of using a conventional method of distinguishing between a healthy person and a diseased person using a temperature threshold value.

FIG. 11 is a graph illustrating the relationship between a healthy person erroneous distinction rate of erroneously identifying a healthy person as a diseased person and a diseased person erroneous distinction rate of erroneously identifying a diseased person as a healthy person, and a temperature threshold value in the conventional method.

FIG. 12 is a graph illustrating the relationship between the healthy person erroneous distinction rate and the diseased person erroneous distinction rate, and the temperature threshold value in a case where the temperature is measured to be lower by 0.5° C.

FIG. 13 is a graph illustrating the relationship between the healthy person erroneous distinction rate and the diseased person erroneous distinction rate, and the temperature threshold value in a case where the temperature is measured to be higher by 0.5° C.

FIG. 14 is a list illustrating distinction results between a healthy person and a diseased person using the temperature pattern according to the first embodiment of the present invention.

FIG. 15 is a diagram illustrating an example of the measurement result of the body surface temperature including the neck part of the subject according to a second embodiment of the present invention.

FIG. 16 is a graph illustrating the temperature patterns of the head, the temples, and the neck of the subject according to the second embodiment of the present invention.

FIG. 17 is a list illustrating distinction results between a healthy person and a diseased person using the temperature pattern according to the second embodiment of the present invention.

FIG. 18 is a block diagram illustrating an internal configuration example of a diseased person distinguishing device according to a third embodiment of the present invention.

FIG. 19 is a block diagram illustrating an internal configuration example of a diseased person distinguishing system according to a fourth embodiment of the present invention.

FIG. 20 is a block diagram illustrating an internal configuration example of a diseased person distinguishing device according to the fourth embodiment of the present invention.

FIG. 21 is a block diagram illustrating an internal configuration example of a diseased person distinguishing device according to a fifth embodiment of the present invention.

### DESCRIPTION OF EMBODIMENTS

Hereinbelow, embodiments of the present invention will be described with reference to the accompanying drawings. In the present specification and the drawings, components having substantially the same function or configuration are labeled with the same reference signs, and redundant description is omitted.

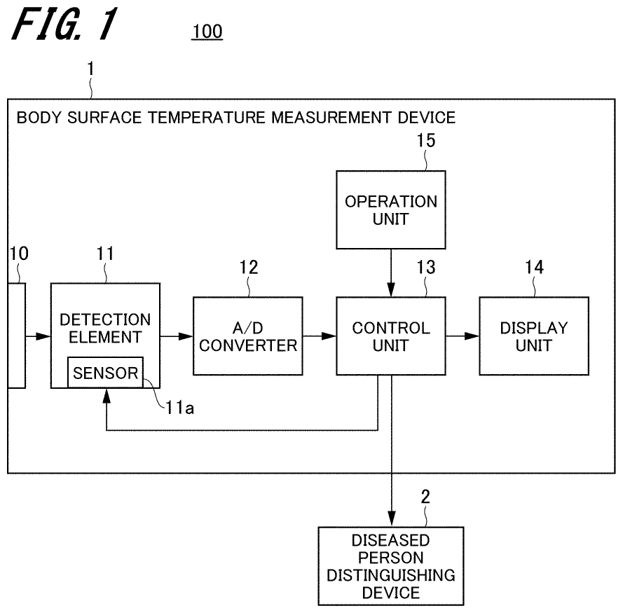

FIG. 1 is a block diagram illustrating an internal configuration example of a diseased person distinguishing system 100.

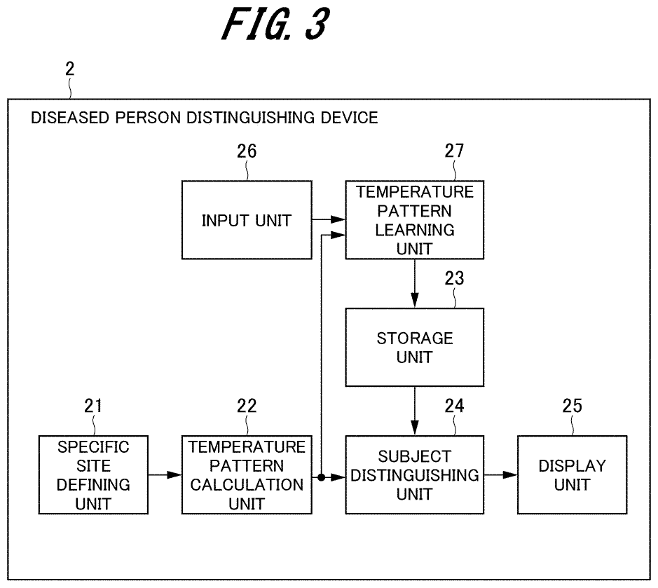

The diseased person distinguishing system 100 includes a body surface temperature measurement device 1 and a diseased person distinguishing device 2. The body surface temperature measurement device 1 and the diseased person distinguishing device are connected by wire or wirelessly. The diseased person distinguishing device 2 acquires a thermal image representing the body surface temperature of a subject measured by the body surface temperature measurement device 1, and determines whether the subject is a diseased person or a healthy person. A detailed configuration example of the diseased person distinguishing device 2 will be described below with reference to FIG. 3.

The body surface temperature measurement device 1 includes a lens 10, a detection element 11, an A/D converter 12, a control unit 13, a display unit 14, and an operation unit 15. The body surface temperature measurement device 1 can measure the body surface temperature of the subject at which the lens 10 is aimed.

The lens 10 condenses infrared light emitted from the subject and forms an image on the detection element 11.

The detection element 11 includes one or a plurality of sensors 11a, and outputs a detection signal in a time division manner for each frame period in synchronization with a vertical synchronization signal input into the detection element 11. The frame rate at which the detection element 11 can output the detection signals per unit time is, for example, 60 frames per second (fps).

In a case where the body surface temperature measurement device 1 is used as a camera, the sensor 11a included in the detection element 11 is a sensor provided per pixel. The sensor 11a detects the intensity of the infrared light formed on the detection element 11 and outputs a detection signal. As the sensor 11a, for example, a microbolometer whose resistance value changes depending on the absolute temperature of the measurement object is used. In the detection element 11, multiple microbolometers, such as 320 in the horizontal direction×240 in the vertical direction, are two-dimensionally arranged. When detecting infrared light, the microbolometer outputs an analog detection signal corresponding to the intensity of the infrared light. However, only one sensor 11a may be used for the detection element 11.

The A/D converter 12 converts the detection signal output from the sensor 11a per pixel of the detection element 11 into digital data, and outputs the digital data to the control unit 13.

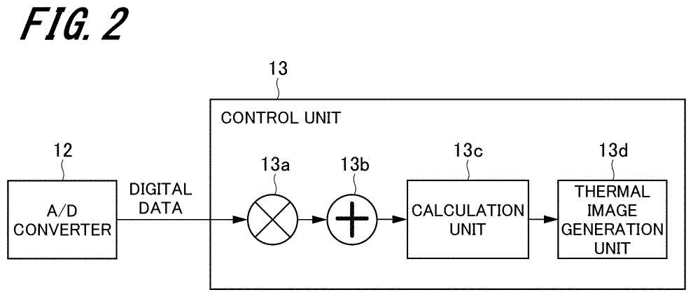

The control unit 13 performs gain correction and offset correction illustrated in FIG. 2 described below on the digital data of the detection signal input from the A/D converter 12, and calculates the absolute temperature of the measurement object. The control unit 13 then performs image processing on the basis of the calculated absolute temperature of the measurement object and outputs obtained image data to the display unit 14. Here, the control unit 13 generates image data of a thermal image in accordance with the operation of the operation unit 15 and outputs the image data to the display unit 14. The control unit 13 also outputs the vertical synchronization signal to the sensor 11a of the detection element 11.

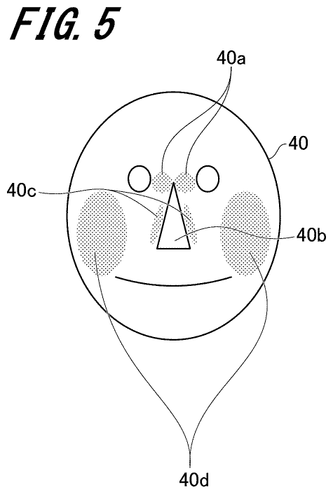

The control unit 13 further outputs temperature data to the diseased person distinguishing device 2. The temperature data includes a thermal image obtained by photographing the entire face of the subject including specific sites on the face of the subject, or the temperatures of the specific sites measured for the respective specific sites. As illustrated in FIG. 5 described below, at least one of the specific sites is, for example, any one of the eye inner corner, the nose tip, the nose side, the cheek, the jaw, the ear, the hand, the head excluding the hair portion, the temple, and the neck of the subject. It is assumed that the positions of the specific sites to be extracted from the thermal image of the face by a specific site defining unit 21 illustrated in FIG. 3 described below are determined in advance.

On the display unit 14, a thermal image indicating the distribution state of the absolute temperatures of the measurement object is displayed on the basis of the image data generated by the control unit 13.

The operation unit 15 is used to input an instruction to cause the control unit 13 to output the absolute temperature of the measurement object. The control unit 13 outputs to the display unit 14 image data to be displayed as a thermal image in accordance with an instruction from the operation unit 15.

Meanwhile, by connecting a personal computer (PC) terminal or the like to the body surface temperature measurement device 1, the display unit 14 may be excluded from the configuration of the body surface temperature measurement device 1. In this case, the control unit 13 outputs to the PC terminal the absolute temperature of the measurement object calculated on the basis of the detection signal input from the detection element 11 for each frame. As a result, the absolute temperature of the measurement object can be displayed on the PC terminal or the like connected to the body surface temperature measurement device 1.

FIG. 2 is a block diagram illustrating an internal configuration example of the control unit 13.

The control unit 13 includes a gain correction unit 13a, an offset correction unit 13b, a calculation unit 13c, and a thermal image generation unit 13d.

The detection signals output from the respective sensors 11a of the detection element 11 are expressed to approximate a linear expression in which a larger value is obtained as the absolute temperature of the measurement object becomes higher. However, the characteristics of the microbolometers corresponding to the respective sensors 11a of the detection element 11 vary, and the linear expression itself deviates from the standard linear expression. Therefore, for example, at the time of factory shipment of the body surface temperature measurement device 1, correction values used for gain correction and offset correction for leveling characteristics of the microbolometers corresponding to the respective sensors 11a are stored in a not-illustrated memory. The control unit 13 then corrects digital data on the basis of the correction values read from the memory.

The gain correction unit 13a of the control unit 13 performs gain correction on the detection signal input from the A/D converter 12 in a state of being converted into digital data. The gain correction unit 13a corrects the gain, that is, the slope of the linear expression.

The offset correction unit 13b performs offset correction on the digital data whose gain has been corrected by the gain correction unit 13a. The offset correction unit 13b corrects the offset, that is, the intercept of the linear expression.

The calculation unit 13c calculates the absolute temperature of the measurement object for each pixel on the basis of the digital data which has been converted from the detection signal and corrected by the gain correction unit 13a and the offset correction unit 13b.

The thermal image generation unit 13d generates image data for displaying a thermal image on the basis of the absolute temperatures of the measurement object calculated for the respective sensors 11a by the calculation unit 13c. The image data generated by the thermal image generation unit 13d is output to the display unit 14 and displayed as a thermal image of the measurement object on the display unit 14.

FIG. 3 is a block diagram illustrating an internal configuration example of the diseased person distinguishing device 2.

As described above, the diseased person distinguishing device 2 determines whether the subject is a diseased person or a healthy person on the basis of information acquired from the body surface temperature measurement device 1. The diseased person distinguishing device 2 includes the specific site defining unit 21, a temperature pattern calculation unit 22, a storage unit 23, a subject distinguishing unit 24, a display unit 25, an input unit 26, and a temperature pattern learning unit 27.

The specific site defining unit 21 defines specific sites in the exposed region of the subject on the basis of the temperature data input from the body surface temperature measurement device 1. The temperature data includes a thermal image obtained as the body surface temperature measurement device 1 photographs the face of the subject. Note that the face of the subject does not necessarily face the front side of the lens 10. Therefore, the specific site defining unit 21 has a function of locating face parts constituting the face on the basis of the thermal image of the face. The specific site defining unit 21 locates the face parts constituting the face on the thermal image using the locating function and defines the specific sites on the thermal image. As described above, in the present embodiment, at least one of the specific sites of the subject is, for example, any one of the eye inner corner, the nose tip, the nose side, the cheek, the jaw, the ear, the hand, the head excluding the hair portion, the temple, and the neck. The specific site defining unit 21 is configured to output the thermal image after locating to the temperature pattern calculation unit 22. However, the specific site defining unit 21 may be configured to output, to the temperature pattern calculation unit 22, a combination or the like of the thermal image before locating and the coordinate group of the specific sites in the image.

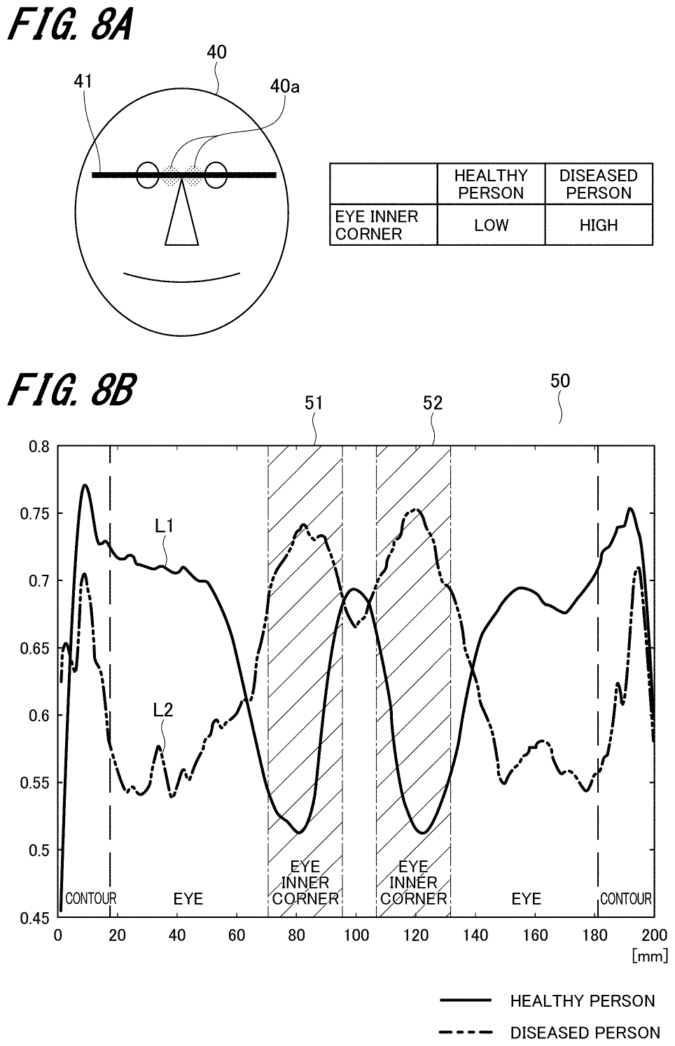

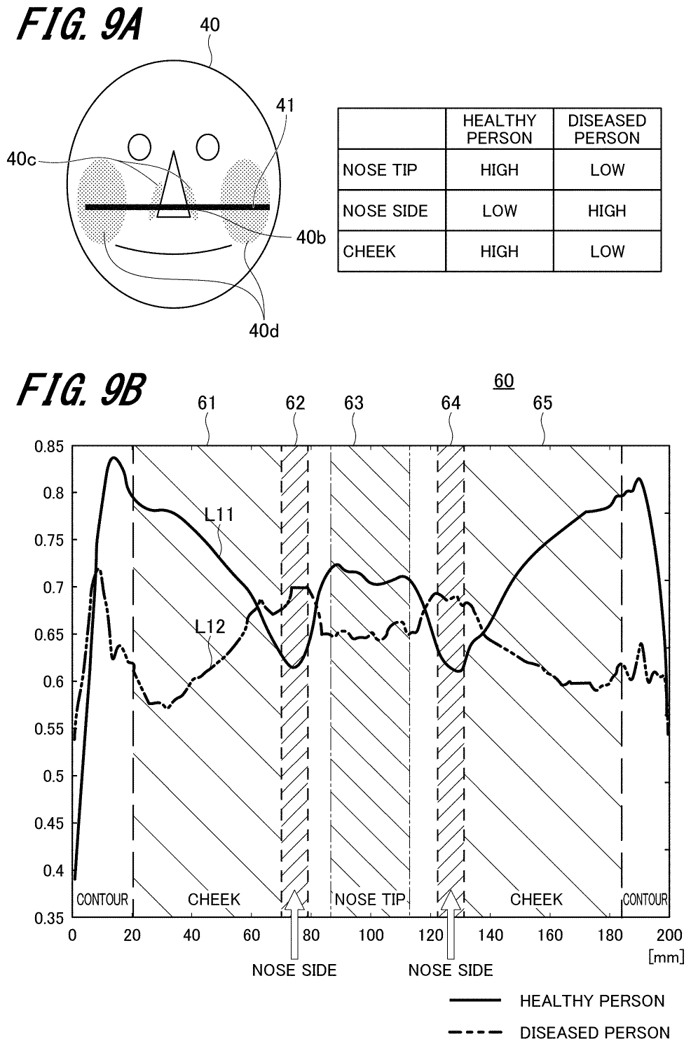

The temperature pattern calculation unit 22 calculates a temperature pattern from the temperatures in the exposed region of the subject on the basis of the temperature data input from the body surface temperature measurement device 1 that measures the body surface temperature of the subject. The temperature pattern is expressed as a group of temperature data collected from a plurality of specific sites of the subject. For example, as illustrated in FIGS. 8 and 9 described below, the temperature pattern is a graph expressing normalized temperature values for the respective specific sites included in a region horizontally crossing the face of the subject, but may be expressed in another form. Also, the temperature pattern may be calculated from the temperatures before normalization instead of the normalized temperature values.

Each specific site includes a plurality of pixels. Therefore, the temperature pattern calculation unit 22 calculates the average value of the values for the plurality of pixels included in the region determined as the specific site as the temperature of the specific site. That is, the temperature pattern calculation unit 22 can extract temperature data from the specific sites defined by the specific site defining unit 21 and derive a temperature pattern of the plurality of specific sites. Note that, as illustrated in FIGS. 8 and 9 described below, the temperature pattern calculation unit 22 calculates normalized temperature values for respective specific sites obtain a temperature pattern. The normalized temperature values calculated by the temperature pattern calculation unit 22 are output to the subject distinguishing unit 24 and the temperature pattern learning unit 27 in association with the plurality of specific sites.

Note that the temperature pattern calculation unit 22 may calculate as the temperature of each specific site the highest value among the values for the plurality of pixels included in the region defined as the specific site, associate the calculated temperature with the specific site, and output the temperature pattern of the specific sites to the subject distinguishing unit 24 and the temperature pattern learning unit 27.

The storage unit 23 stores a learned model obtained by learning temperature patterns of subjects. The learned model is used by the subject distinguishing unit 24 to determine that the subject is either a diseased person or a healthy person on the basis of the temperature pattern of the subject. In a case where the diseased person distinguishing device 2 has a learning function, the learned model is one learned by the temperature pattern learning unit 27 described below. However, in a case where the diseased person distinguishing device 2 does not have a learning function, the learned model is one prepared in advance in the storage unit 23.

The subject distinguishing unit 24 applies the learned model learned in advance read from the storage unit 23 to the calculated temperature pattern to determine whether the subject is a diseased person or a healthy person. The distinction result of the subject distinguishing unit 24 is output to the display unit 25.

The display unit 25 displays a distinction result including the possibility that the subject is a diseased person. For example, a text message indicating that the subject is a diseased person or a healthy person is displayed on the display unit 25 as a distinction result. Note that a notification unit that generates an alarm may be provided instead of the display unit 25. The notification by means of an alarm is performed, for example, by the notification unit turning on a lamp or transmitting an e-mail to an administrator of the facility.

Next, an operation example of the input unit 26 and the temperature pattern learning unit 27, which are responsible for processing for generating the learned model, will be described. Note that the processing for generating the learned model is not always performed.

The input unit 26 receives a user distinction result as to whether the subject is a healthy person or a diseased person input by the user who uses the diseased person distinguishing device 2. For example, in a case where the subject is actually a healthy person, “healthy person” is input through the input unit 26. On the other hand, in a case where the subject is actually a diseased person, “diseased person” is input through the input unit 26.

The user inputs the user distinction result through the input unit 26 to correct the learned model. The input user distinction result is either “healthy person” or “diseased person” described above. Note that, when the user inputs the user distinction result through the input unit 26, the user does not always need to check the output of the display unit 25 and determine whether the distinction result of the subject distinguishing unit 24 is correct. The temperature pattern learning unit 27 corrects the learned model by being given the temperature pattern given from the temperature pattern calculation unit 22 and a so-called correct answer as to whether the temperature pattern should be identified as that for a healthy person or a diseased person.

The temperature pattern learning unit 27 generates a learned model learned by means of a machine learning model on the basis of the user distinction result input from the input unit 26 and the temperature pattern which is the output of the temperature pattern calculation unit 22 and used in the subject distinguishing unit 24. The learned model is data in which the learning results of the temperature pattern learning unit 27 are accumulated.

The temperature pattern learning unit 27 updates the learned model by means of machine learning, and stores the updated learned model in the storage unit 23. The learned model is updated regardless of whether the result is correct or incorrect. That is, the learned model is corrected and updated regardless of whether the output of the subject distinguishing unit 24 and the actual situation match or do not match. As the temperature pattern learning unit 27 repeats learning, the learned model is gradually corrected to an appropriate learned model and updated. Thereafter, the subject distinguishing unit 24 applies the updated learned model to the temperature pattern calculated by the temperature pattern calculation unit 22 to determine whether the subject is a diseased person or a healthy person.

Next, a hardware configuration of a computing machine 30 constituting each device of the diseased person distinguishing system 100 will be described.

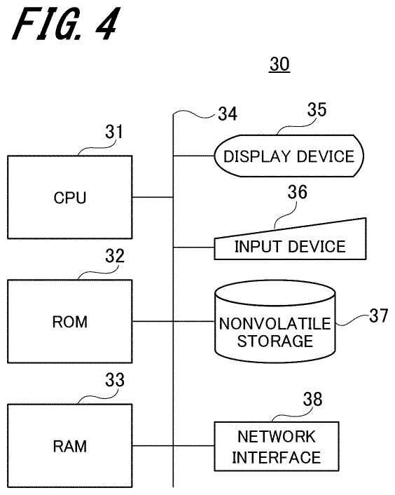

FIG. 4 is a block diagram illustrating a hardware configuration example of the computing machine 30. The computing machine 30 is used as, for example, hardware constituting the body surface temperature measurement device 1 and the diseased person distinguishing device 2.

The computing machine 30 is hardware used as a so-called computer. The computing machine 30 includes a central processing unit (CPU) 31, a read only memory (ROM) 32, and a random access memory (RAM) 33 each connected to a bus 34. The computing machine 30 also includes a display device 35, an input device 36, a nonvolatile storage 37, and a network interface 38.

The CPU 31 reads program code of software that fulfills respective functions according to the present embodiment from the ROM 32, loads the program code into the RAM 33, and executes the program code. Variables, parameters, and the like generated during arithmetic processing of the CPU 31 are temporarily written into the RAM 33, and these variables, parameters, and the like are read by the CPU 31 as needed. In the body surface temperature measurement device 1, the function of the control unit 13 is mainly fulfilled by the CPU 31. In the diseased person distinguishing device 2, the functions of the specific site defining unit 21, the temperature pattern calculation unit 22, the subject distinguishing unit 24, and the temperature pattern learning unit 27 are fulfilled by the CPU 31.

The display device 35 is, for example, a liquid crystal display monitor, and displays the result of processing performed in the computing machine 30, a thermal image, and the like to the user. For example, the display device 35 is used for the display unit 14 of the body surface temperature measurement device 1 and the display unit 25 of the diseased person distinguishing device 2.

As the input device 36, an operation key or an operation button is used, for example, with which the user can perform predetermined operation inputs and instructions. For example, the input device 36 is used for the operation unit. 15 of the body surface temperature measurement device 1 and the input unit 26 of the diseased person distinguishing device 2.

As the nonvolatile storage 37, a hard disk drive (HDD), a solid state drive (SSD), a flexible disk, an optical disk, a magneto-optical disk, a CD-ROM, a CD-R, a magnetic tape, or a nonvolatile memory is used, for example. The nonvolatile storage 37 has recorded therein not only an operating system (OS) and various parameters but also a program for causing the computing machine 30 to function. The ROM 32 and the nonvolatile storage 37 permanently record a program, data, and the like required for operation of the CPU 31, and are used as examples of a computer-readable non-transitory recording medium storing a program executed by the computing machine 30. For example, the nonvolatile storage 37 is used for the storage unit 23 of the diseased person distinguishing device 2.

As the network interface 38, a network interface card (NIC) is used, for example, and various data can be transmitted and received between devices via a local area network (LAN), a dedicated line, or the like connected to a terminal of the NIC. For example, the network interface 38 is used for a not-illustrated communication unit that communicably connects the body surface temperature measurement device 1 and the diseased person distinguishing device 2 to each other.

FIG. 5 is an explanatory diagram illustrating positions of the specific sites.

FIG. 5 illustrates a thermal image 40 of the face of the subject and the positions of the specific sites. As the specific sites, for example, eye inner corners 40a, a nose tip 40b, nose sides 40c, and cheeks 40d are illustrated. The thermal image 40 of the face of the subject can also be displayed as a black-and-white image in which a higher temperature portion is closer to white and a lower temperature portion is closer to black.

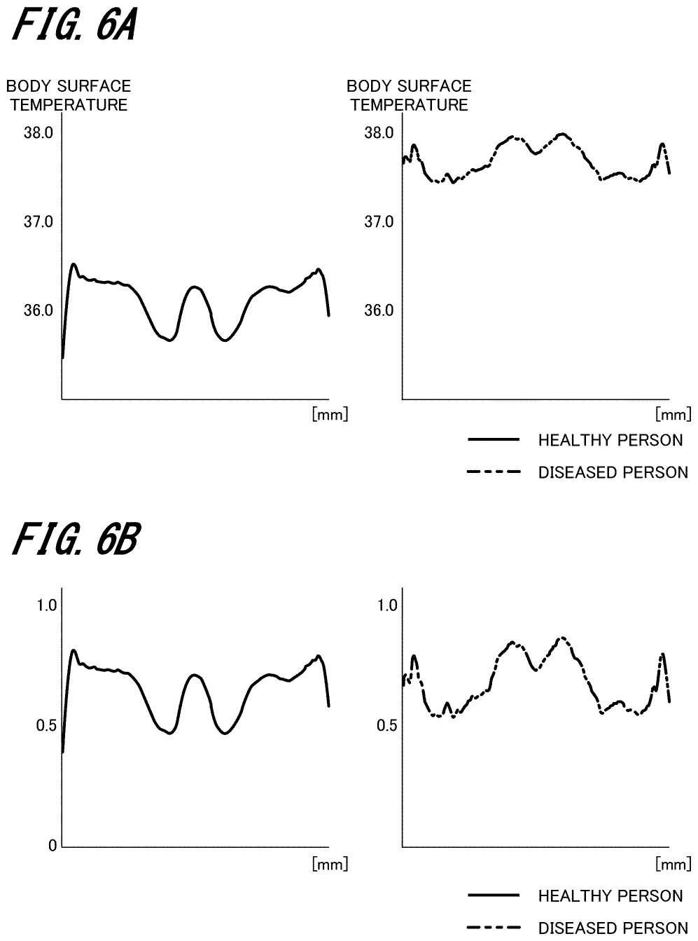

FIG. 6 is a diagram for explaining how the temperature pattern of the entire face including the specific sites is normalized.

FIG. 6A illustrates graphs in which the body surface temperatures of a plurality of healthy persons and a plurality of diseased persons obtained by photographing by means of the body surface temperature measurement device 1 are averaged for each part. The horizontal axis of each graph represents the position [mm] of the sites along the line crossing certain specific sites of the healthy person or the diseased person, and the vertical axis represents the body surface temperature [° C.] of the healthy person or the diseased person. Also, the body surface temperature of the healthy person is represented by the solid line, and the body surface temperature of the diseased person is represented by the two-dot chain line. The graphs illustrated on the upper side of FIG. 6 are used as examples in which the temperature values of the healthy person and the diseased person are clearly different in order to describe the temperature patterns according to the present embodiment.

In general, the body surface temperature of a diseased person tends to be higher than the body surface temperature of a healthy person. For example, while the temperature of the face surface of a healthy person is in the range of 36.0° C. to 37.0° C., the temperature of the face surface of a diseased person is in the range of 37.0° C. to 38.0° C. Hence, when the temperature of the entire face of a diseased person rises, and the entire thermal image of the face becomes white, it is difficult to compare the thermal image of the face of the diseased person with that of a healthy person. Therefore, each graph is normalized and expressed so that a characteristic temperature pattern can be obtained from the thermal image of the face of each of the diseased person and the healthy person.

FIG. 6B illustrates normalized body surface temperatures of the healthy person and the diseased person. Each of these graphs is obtained by normalizing the body surface temperature of the healthy person or the diseased person illustrated on the upper side of FIG. 6 within a range of 0 to 1.0. By normalizing the body surface temperatures of the healthy person and the diseased person, temperature patterns characteristic of the healthy person and the diseased person are found.

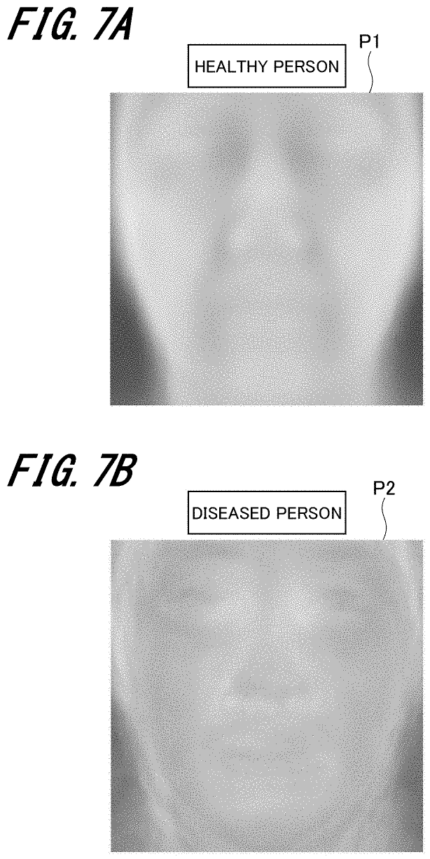

FIG. 7 is a diagram illustrating display examples of normalized thermal images of the faces of a healthy person and a diseased person.

A thermal image P1 of the face of a healthy person is displayed in FIG. 7A, and a thermal image P2 of the face of a diseased person is displayed in FIG. 7B. The thermal image P1 of the face of the healthy person is an image obtained by normalizing the thermal image of the face of the healthy person obtained by photographing by means of the body surface temperature measurement device 1. Similarly, the thermal image P2 of the face of the diseased person is an image obtained by normalizing the thermal image of the face of the diseased person obtained by photographing by means of the body surface temperature measurement device 1. In the normalized thermal images P1 and P2 of the faces, a portion having a high temperature is displayed in white, and a portion having a low temperature is displayed in black.

By normalizing the temperature ranges measurable by the body surface temperature measurement device 1 to have the same range from a low temperature to a high temperature, characteristic temperature patterns are obtained in the thermal images P1 and P2 of the faces. Therefore, based on the temperature pattern obtained by normalizing the thermal image of the face, it can be determined whether the subject is a healthy person or a diseased person.

Next, an example of the temperature pattern will be described with reference to FIGS. 8 and 9. In the present embodiment, a temperature pattern in a region horizontally crossing the thermal image 40 of the face of the subject will be described.

FIG. 8 is an explanatory diagram illustrating an example of a temperature pattern in the thermal image 40 of the face on a horizontal line 41 passing through the eye inner corners 40a.

FIG. 8A illustrates the horizontal line 41 passing through the eye inner corners 40a of the thermal image 40 of the face of the subject and a list illustrating the difference between a healthy person and a diseased person in terms of the normalized temperature value in the specific site. The horizontal line 41 indicates a position where the temperature pattern calculation unit 22 has scanned the thermal image 40 of the face to calculate the temperature pattern of the thermal image 40 of the face.

FIG. 8B illustrates a temperature pattern 50 in the thermal images 40 of the faces of the subjects measured along the horizontal line 41. The horizontal axis of the temperature pattern 50 represents a position [mm] of each part in the thermal image 40 of the face, and the vertical axis represents a value obtained by normalizing the temperature (normalized temperature value). The temperature pattern of a healthy person is expressed as a solid line chart L1, and the temperature pattern of a diseased person is expressed as a two-dot chain line chart L2.

The temperature pattern 50 indicates that, in each of ranges 51 and 52 representing the eve inner corners 40a, the normalized temperature value of the eye inner corner 40a of the diseased person is higher than the normalized temperature value of the eye inner corner 40a of the healthy person. Therefore, the list illustrated in FIG. 8A indicates that the normalized temperature value of the eye inner corner 40a of the healthy person is low while that of the diseased person is high.

FIG. 9 is a diagram illustrating an example of a temperature pattern in the thermal image 40 of the face on the horizontal line 41 passing through the nose tip 40b.

FIG. 9A illustrates the horizontal line 41 passing through the nose tip 40b of the thermal image 40 of the face of the subject and a list illustrating the difference between a healthy person and a diseased person in terms of the normalized temperature values in the specific sites.

FIG. 9B illustrates a temperature pattern 60 in the thermal images 40 of the faces of the subjects measured along the horizontal line 41. The horizontal axis of the temperature pattern 60 represents a position [mm] of each part in the thermal image 40 of the face, and the vertical axis represents a value obtained by normalizing the temperature. The temperature pattern of a healthy person is expressed as a solid line chart L11, and the temperature pattern of a diseased person is expressed as a two-dot chain line chart L12.

The temperature pattern 60 indicates that, in a range 63 representing the nose tip 40b, the normalized temperature value of the nose tip 40b of the healthy person is higher than the normalized temperature value of the nose tip 40b of the diseased person. The temperature pattern 60 also indicates that, in each of ranges 62 and 64 representing the nose side 40c, the normalized temperature value of the nose side 40c of the diseased person is higher than the normalized temperature value of the nose side 40c of the healthy person. The temperature pattern 60 further indicates that, in each of ranges 61 and 65 representing the cheek 40d, the normalized temperature value of the cheek 40d of the healthy person is higher than the normalized temperature value of the cheek 40d of the diseased person.

Therefore, the list illustrated in FIG. 9A indicates that the normalized temperature value of the nose tip 40b of the healthy person is high while the normalized temperature value of the nose tip 40b of the diseased person is low. Similarly, the list indicates that the normalized temperature value of the nose side 40c of the healthy person is low while the normalized temperature value of the nose side 40c of the diseased person is high, and that the normalized temperature value of the cheek 40d of the healthy person is high while the normalized temperature value of the cheek 40d of the diseased person is low.

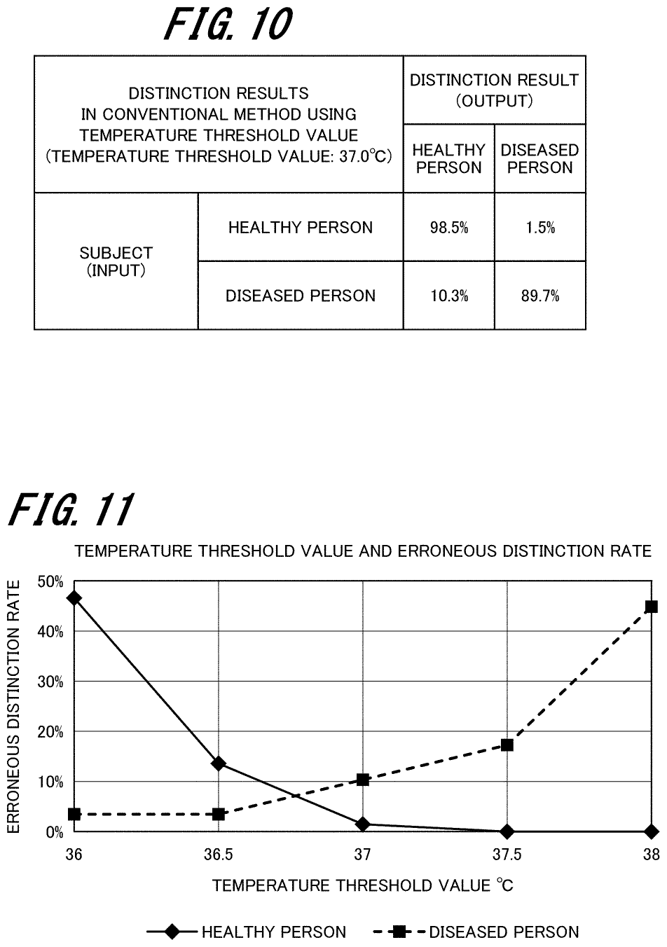

FIG. 10 is a list illustrating an example of distinction results in the case of using a conventional method of distinguishing between a healthy person and a diseased person using a temperature threshold value. In FIG. 10, the user distinction result previously input by the user is expressed as “subject (input)”. The distinction result of the subject output in the conventional method is expressed as “distinction result (output)”. In the items of “subject (input)” and “distinction result (output)”, items of “healthy person” and “diseased person” are provided, respectively.

Here, an example of determining that the subject is either a healthy person or a diseased person using 37.0° C. as a temperature threshold value will be described. In the conventional method, since the temperature threshold value is set, 98.5% of the healthy people are correctly identified as healthy people, but 1.5% of the healthy people are erroneously identified as diseased people. Also, in the conventional method, 89.7% of the diseased people are correctly identified as diseased people, but 10.3% of the diseased people are erroneously identified as healthy people.

On the other hand, there are individual differences among general infrared cameras that can be used as the body surface temperature measurement device, and the temperature value may be measured to be lower or higher than the actual temperature value by about ±0.5° C. Also, the temperature value can be measured to be lower or higher than the actual temperature value depending on the environmental conditions such as outside air temperature. Therefore, a threshold value properly set at one location at one time could not be an appropriate threshold value at another location at another time.

FIG. 11 is a graph illustrating a state in which a healthy person erroneous distinction rate of erroneously identifying a healthy person as a diseased person and a diseased person erroneous distinction rate of erroneously identifying a diseased person as a healthy person vary depending on the set temperature threshold value.

In the conventional method, in a case where the temperature threshold value is lowered, the diseased person erroneous distinction rate of erroneously identifying a diseased person as a healthy person decreases, but the healthy person erroneous distinction rate of erroneously identifying a healthy person as a diseased person increases. Conversely, in a case where the temperature threshold value is raised, the healthy person erroneous distinction rate decreases, but the diseased person erroneous distinction rate increases.

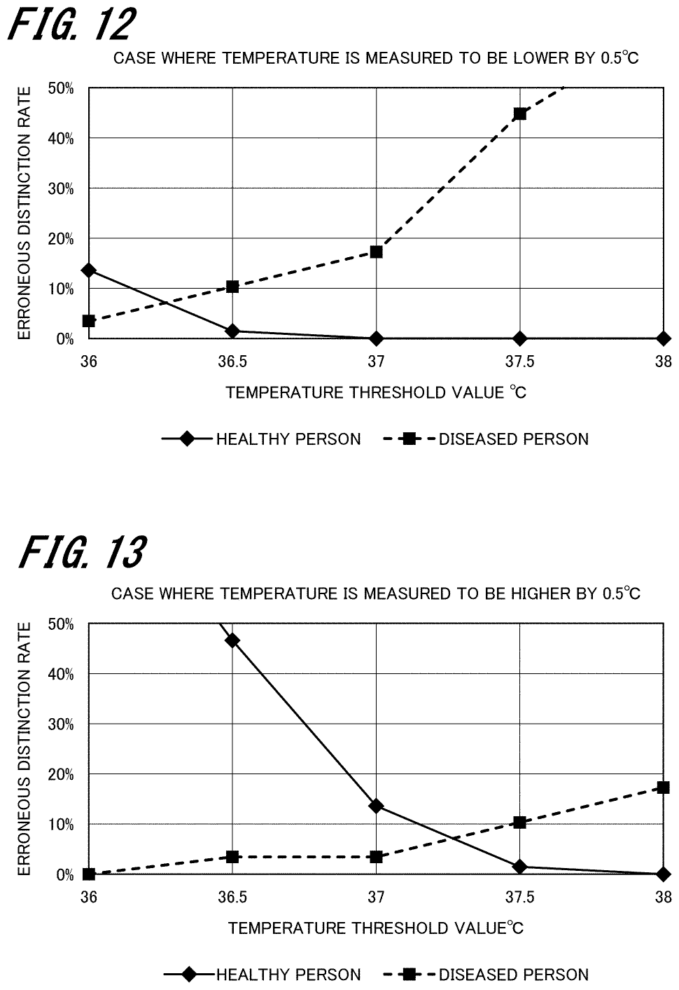

FIG. 12 is a diagram illustrating the erroneous distinction rates in a case where the temperature is measured to be lower by 0.5° C.

In a case where the conventional method is used with the temperature threshold value remaining 37.0° C., the diseased person erroneous distinction rate increases to 17.2%.

FIG. 13 is a diagram illustrating the erroneous distinction rates in a case where the temperature is measured to be higher by 0.5° C.

In a case where the conventional method is used with the temperature threshold value remaining 37.0° C., the healthy person erroneous distinction rate increases to 13.6%.

In this manner, in the conventional method, a process for appropriately setting a temperature threshold value is required in order to appropriately distinguish between a healthy person and a diseased person, and it has been said that experience is required in order to appropriately set a temperature threshold value.

Also, conventionally, it has not been possible to accurately determine that a subject is a diseased person. Hence, in a case where the rate at which a subject is identifies as a healthy person although the subject is actually a diseased person is high, the diseased person identified as a healthy person may enter the facility.

Also, in a diseased person distinguishing system using a conventional infrared camera, an alarm is generated when a maximum temperature value in a thermal image of a face exceeds a temperature threshold value. However, there are large individual differences among body surface temperatures. Hence, in a case where a fixed temperature threshold value is used, even a healthy person is erroneously identified as a diseased person, and frequent alarms sometimes cause confusion in the operation site. In a case where the temperature threshold value is excessively raised in order to avid the frequent alarms, this causes a problem in which a diseased person cannot be screened out and is allowed to pass. Furthermore, the body surface temperature is easily affected by the outside air temperature, and the user has to change the temperature threshold value into an appropriate value in accordance with the outside air temperature, which takes time and effort.

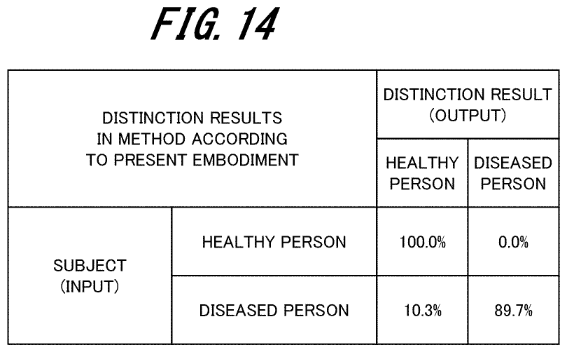

FIG. 14 is a list illustrating distinction results between a healthy person and a diseased person using the temperature pattern according to the first embodiment of the present invention. In FIG. 14 as well, the user distinction result previously input by the user is expressed as “subject (input)”. The distinction result of the subject output in the method according to the first embodiment is expressed as “distinction result (output)”.

In a case where the method according to the first embodiment is used, setting of a temperature threshold value, which has conventionally been performed, is not required. As for comparison in terms of the same experimental data, the diseased person erroneous distinction rate is 10.3%, which is equivalent, but the healthy person erroneous distinction rate is 0%. Therefore, by using the method according to the first embodiment, superior results were obtained as compared with the case of distinction between a healthy person and a diseased person using an appropriate temperature threshold value in the conventional method.

Therefore, by using the method according to the present embodiment, the accuracy of distinction between a healthy person and a diseased person is improved as compared with the conventional method. Consequently, the diseased person distinguishing device 2 can reliably determine that the subject is a diseased person and restrict entry into the facility.

In the diseased person distinguishing system 100 according to the first embodiment described above, attention is paid to the fact that the temperature patterns of the faces of a diseased person and a healthy person are different, and a diseased person and a healthy person can be distinguished using a learned model learned by means of a machine learning model. Here, the temperature patterns of the faces of the diseased person and the face of the healthy person are different, for example, at the eye inner corner, the nose tip, the nose side, and the cheek. Therefore, the diseased person distinguishing device 2 can calculate the temperature pattern of each site such as the eye inner corner, the nose tip, the nose side, and the cheek on the basis of measurement by the body surface temperature measurement device 1, and easily and accurately distinguish between a diseased person and a healthy person by applying the learned model learned in advance. Consequently, it is possible to manage the access of the subject to the facility using the distinction result.

As compared with the conventional method for distinguishing between a diseased person and a healthy person using a temperature threshold value, in the present embodiment, by applying the learned model to the calculated temperature pattern, the diseased person distinguishing device 2 can distinguish between a diseased person and a healthy person regardless of the individual differences in the body surface temperatures of the subjects or the outside air temperature of the place where the subjects stay, and the erroneous determination of the diseased person is reduced. Also, while it has been difficult to set an appropriate temperature threshold value depending on the sex and age of the subject, in the present embodiment, differences in temperature patterns between a diseased person and a healthy person are used. Therefore, a delicate setting of a temperature threshold value or the like becomes unnecessary, and the operation becomes easy.

Also, by learning the temperature pattern by means of the machine learning model and using the accumulated learning result as the learned model, it is possible to distinguish between a diseased person and a healthy person in terms of various diseases. It is also possible to generate different learned models depending on whether the subject is a child or an adult, the difference in gender, the difference in nationality, and the like. It is further possible to generate different learned models depending on the conditions of the operation place such as temperature. By doing so, the accuracy with which the diseased person distinguishing device 2 distinguishes between a diseased person and a healthy person is improved.

Also, even before onset, that is, before the body surface temperature of the subject rises, the temperature change indicated by the temperature pattern tends to occur as it occurs after onset. Therefore, by applying the learned model to the temperature pattern, the diseased person distinguishing device 2 can identify even a subject before onset as a diseased person. In this manner, the diseased person distinguishing device 2 can determine that the subject is a diseased person with high probability. The diseased person distinguishing device 2 can display on the display unit 25 the possibility that the subject before onset is a diseased person to alert the user.

Also, a plurality of parts of the face of the subject are defined as specific sites, and relative changes in temperature at the specific sites of a diseased person and a healthy person are used as temperature patterns. Also, the temperature pattern at the specific sites of the subject only needs to be clarified. Hence, even in a case where a part of the face is covered with a cover such as hair, a mask, and glasses, and where the temperature of the part covered with the cover cannot be acquired, the diseased person distinguishing device 2 can calculate a temperature pattern from the other uncovered specific sites and distinguish between a diseased person and a healthy person.

Also, the temperature patterns of the faces of a diseased person and a healthy person are updated by the temperature pattern learning unit 27. Therefore, sites other than the eye inner corner, the nose tip, the nose side, and the cheek are selected as the specific sites, and the learned model is updated by the temperature pattern at the selected specific sites. The diseased person distinguishing device 2 can identify a diseased person affected with various infectious diseases other than the influenza virus or having poor physical condition using the updated learned model.

Note that the temperature pattern is not only based on the normalized temperature value for each specific site included in the region horizontally crossing the face of the subject. For example, the temperature pattern may be a temperature pattern based on a normalized temperature value for each specific site included in a region vertically or obliquely crossing the face of the subject. Also, the temperature pattern may be obtained from a combination of specific sites that are not included in a region crossing the face of the subject straight. For example, a temperature pattern may be obtained from a combination of the eye inner corners 40a illustrated in FIG. 8 and the cheeks 40d illustrated in FIG. 9, and the subject may be subject to distinction by using the temperature pattern of these specific sites.

Also, in order to explain that each of the temperature patterns among the specific sites has characteristics, an example in which the body surface temperatures of a healthy person and a diseased person are normalized is given using FIGS. 6 to 9. However, the normalization processing may be dispensed with in the actual diseased person distinguishing process and learning process in the diseased person distinguishing device 2.

Also, the diseased person distinguishing device 2 may also use the conventional processing for identifying a subject whose body surface temperature is equal to or higher than a temperature threshold value as a diseased person.

Also, the temperature pattern learning unit 27 may learn not only the relation among the body surface temperatures at the respective specific sites but also the body surface temperatures themselves of the subject. Therefore, the temperature pattern learning unit 27 can learn the absolute value of the body surface temperature of the subject. The subject distinguishing unit 24 can also use the absolute value of the body surface temperature as a criterion for determining whether the subject is a healthy person or a diseased person.

### Second Embodiment

The diseased person distinguishing device 2 according to the first embodiment described above learns the temperature distribution of the face and generates the learned model mainly focusing on the face of the subject on the assumption that the entire face of the subject is exposed. However, the subject may wear glasses, wear an eyepatch, or wear a mask for infectious disease countermeasures. For this reason, in a case where the area in which a part of the specific sites of the subject is covered with a cover such as glasses, a mask, and an eyepatch is large, the temperature distribution of the face may not accurately be obtained. In this case, is expected that it will be difficult for the diseased person distinguishing device 2 to determine whether the subject is a healthy person or a diseased person.

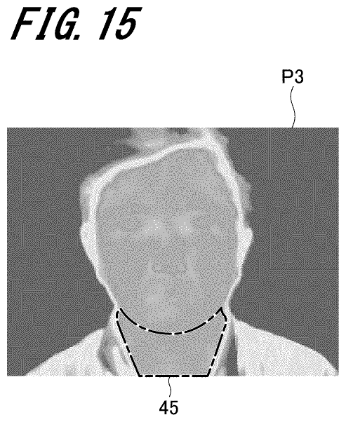

Therefore, the diseased person distinguishing device according to a second embodiment determines whether the subject is a healthy person or a diseased person on the basis of not only the body surface temperature of the face surface of the subject but also that of the neck part. Hereinbelow, the diseased person distinguishing device according to the second embodiment of the present invention will be described with reference to FIGS. 15 to 17.

The configuration of the body surface temperature measurement device 1 according to the second embodiment is the same as the configuration of the body surface temperature measurement device 1 according to the first embodiment.

The control unit 13 (refer to FIG. 1) according to the second embodiment outputs temperature data to the diseased person distinguishing device 2. The temperature data includes a thermal image obtained by photographing the entire face and the neck of the subject including specific sites on the face of the subject, or the temperatures of the specific sites measured for the respective specific sites. Here, the specific sites according to the second embodiment include a site that is less affected by a cover such as a mask and glasses. For example, a site, such as the neck, through which the artery passes, or a part close to the heart is selected as the specific site. Therefore, at least one of the specific sites according to the second embodiment is any one, including at least the neck, out of the eye inner corner, the nose tip, the nose side, the cheek, the jaw, the ear, the hand, the head excluding the hair portion, the temple, and the neck. Hence, in the second embodiment, temperature data representing the temperature of the neck is included in a group of temperature data.

Also, the specific site defining unit 21 defines specific sites in the exposed region of the subject on the basis of the temperature data input from the body surface temperature measurement device 1. The temperature data includes a thermal image obtained as the body surface temperature measurement device 1 photographs the face and the neck of the subject. Here, the specific site defining unit 21 according to the second embodiment needs to define the neck of the subject as a specific site. However, in a case where the subject wears clothes with a collar, it is expected that only a part of the front of the neck is visible. In this case, a measurement region is created in the portion not covered with the collar (the lower side of the front side of the face), and the maximum value in the measurement region is acquired as the temperature of the neck.

Therefore, the specific site defining unit 21 determines the front side of the face using an algorithm capable of detecting the face and the neck only when the face of the subject faces the front. At this time, the specific site defining unit 21 locates face parts (for example, the contour of the face and the jaw) constituting the front side of the face on the basis of the thermal image. Further, the specific site defining unit 21 determines that the face of the subject faces the front in the same direction as the direction in which the body surface temperature measurement device 1 performs photographing, and defines the specific sites from the thermal image. At this time, the specific site defining unit 21 regards the temperature value in the measurement region created under the face as the temperature of the neck.

FIG. 15 is a diagram illustrating an example of the measurement result of the body surface temperature including the neck part of the subject. In FIG. 15, a thermal image P3 of the subject is displayed. It is assumed that the body surface temperature of the subject is measured in a state where no part of the face is covered with a cover such as a mask. The thermal image P3 indicates that the body surface temperatures of the face and the neck of the subject are substantially the same.

The diseased person distinguishing device 2 according to the second embodiment focuses on the head-and-neck area from the part as high as the height of the clavicle to the forehead of the subject in order to distinguish between a healthy person and a diseased person. Subsequently, when detecting that the face of the subject faces the front, the specific site defining unit 21 sets a measurement region 45 between the clavicle and the jaw of the subject. The specific site defining unit 21 then acquires the temperature in the measurement region 45 as the temperature of the neck. The specific site defining unit 21 also defines specific sites exposed from the face of the subject that is not covered with a cover.

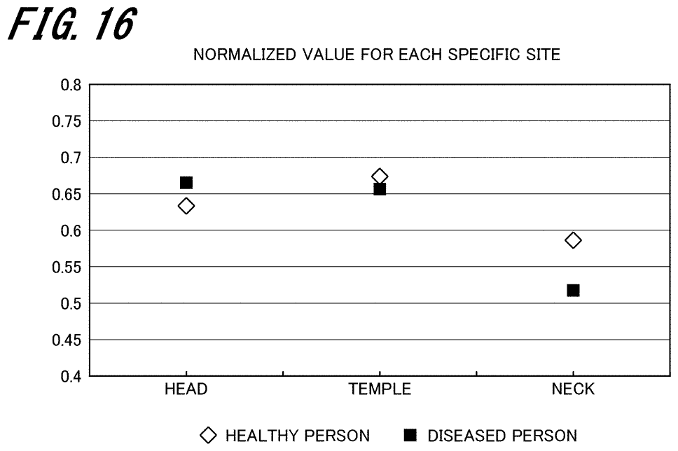

FIG. 16 is a graph illustrating the temperature patterns of the head, the temples, and the neck of the subject. In this graph, the horizontal axis represents the specific sites (the head, the temples, and the neck) of the subject, and the vertical axis represents the normalized value for each specific site. As already described with reference to the graph in FIG. 6, the body surface temperature for each specific site is normalized within a range of 0 to 1.0.

The graph in FIG. 16 shows that the normalized value of the healthy person is lower than the normalized value of the diseased person in the head (for example, the forehead) of the subject.

The graph also shows that the normalized value of the healthy person and the normalized value of the diseased person are substantially equal in the temples of the subject.

The graph also shows that the normalized value of the healthy person is higher than the normalized value of the diseased person in the neck of the subject.

In this manner, in the head-and-neck area from the part as high as the height of the clavicle to the forehead of the subject, the temperature pattern expressed by the normalized values of the healthy person is clearly different from the temperature pattern expressed by the normalized values of the diseased person. In addition, since the normalized values are particularly different in the neck portion of the subject, the distinction accuracy may probably be improved as compared with the case of distinguishing between a healthy person and a diseased person only by the normalized values of the head and the temples.

Therefore, as illustrated in FIG. 3, the temperature pattern calculation unit 22 extracts temperature data from the specific sites including the neck defined by the specific site defining unit 21 on the basis of the temperature data input from the body surface temperature measurement device 1, and derives the temperature pattern of the plurality of specific sites expressed in the graph in FIG. 16. This temperature pattern is output to the subject distinguishing unit 24 and the temperature pattern learning unit 27.

The subject distinguishing unit 24 applies the learned model learned in advance read from the storage unit 23 to the calculated temperature pattern to determine whether the subject is a diseased person or a healthy person. The distinction result of the subject distinguishing unit 24 is output to the display unit 25.

The temperature pattern learning unit 27 generates a learned model learned by means of a machine learning model on the basis of the user distinction result input from the input unit 26 and the temperature pattern which is the output of the temperature pattern calculation unit 22 and used in the subject distinguishing unit 24. The temperature pattern learning unit 27 then updates the learned model stored in the storage unit 23.

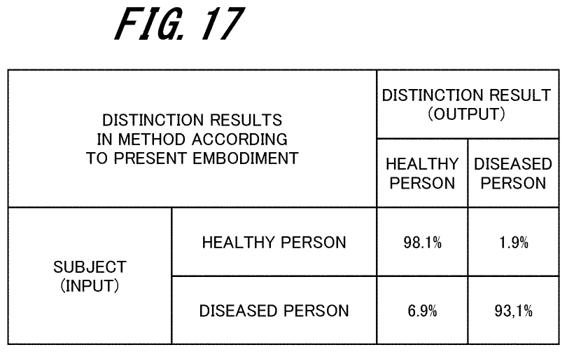

FIG. 17 is a list illustrating distinction results between a healthy person and a diseased person using the temperature pattern according to the second embodiment of the present invention. In FIG. 17 as well, the user distinction result previously input by the user is expressed as “subject (input)”. The distinction result of the subject output in the method according to the second embodiment is expressed as “distinction result (output)”.

In a case where the method according to the second embodiment is used, setting of a temperature threshold value, which has conventionally been performed, is not required. Then, as compared with the conventional method using the temperature threshold value illustrated in FIG. 10, the diseased person erroneous distinction rate decreases from 10.3% to 6.9%, but the healthy person erroneous distinction rate changes from 1.5% to 1.9%, which are almost. the same values. However, the diseased person distinction rate increases from 89.7% to 93.1%. Therefore, by using the method according to the second embodiment, superior results can be obtained as compared with the conventional method for distinguishing between a healthy person and a diseased person using a temperature threshold value. That is, by using the method according to the second embodiment, the accuracy of distinction between a healthy person and a diseased person is improved as compared with the conventional method. Consequently, the diseased person distinguishing device 2 can reliably determine that the subject is a diseased person and restrict entry into the facility.

In the body surface temperature measurement device 1 according to the second embodiment described above, a machine learning model capable of reducing the influence of a cover such as a mask and glasses is created by using the distribution of the normalized temperature values of the specific sites including at least the neck as well as the face as the data for learning. By utilizing the machine learning model learned in advance, the body surface temperature measurement device 1 can identify the subject as either a healthy person or a diseased person with high accuracy even in a case where the specific sites of the face of the subject covered differ per subject depending on the shape of the cover.

Also, specific sites other than the neck may be defined. For example, the finger of the hand of the subject is assumed as the specific site. Any exposed site of the body of the subject can be selected as a candidate for the specific site.

In the body surface temperature measurement device 1 according to the first embodiment as well, the temperature pattern is not limited to the temperature pattern of the normalized temperature values for the respective specific sites included in a region horizontally crossing the face image of the subject, but may be another temperature pattern. For example, similarly to the method according no the second embodiment, the body surface temperature measurement device 1 according to the first embodiment may identify the subject as either a healthy person or a diseased person with high accuracy using a temperature pattern found from a combination of normalized temperature values or temperatures of various sites.

### Third Embodiment

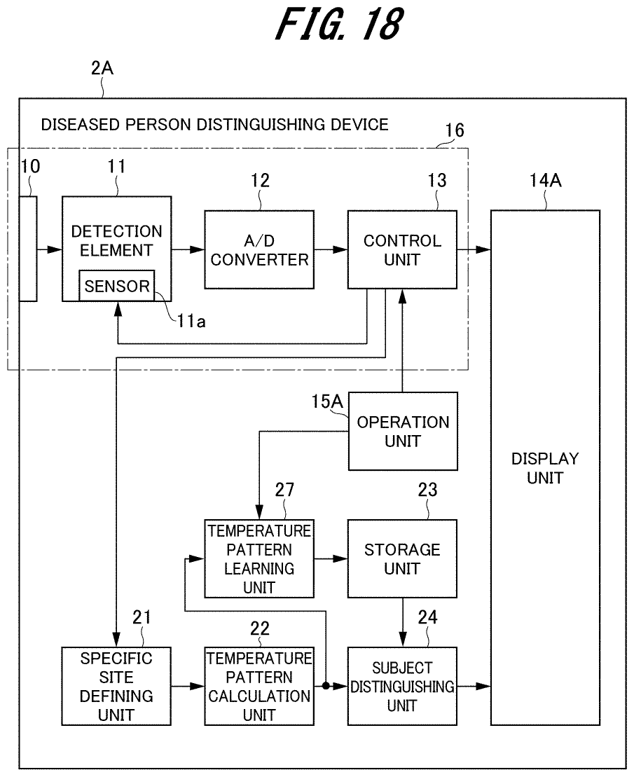

Next, a diseased person distinguishing device according to a third embodiment of the present invention will be described with reference to FIG. 18.

FIG. 18 is a block diagram illustrating an internal configuration example of a diseased person distinguishing device 2A.

The diseased person distinguishing device 2A is obtained by combining and integrating the functional units of the body surface temperature measurement device 1 illustrated in FIG. 1 and the functional units of the diseased person distinguishing device 2 illustrated in FIG. 3. Here, the lens 10, the detection element 11 and the sensor 11a, the A/D converter 12, and the control unit 13 are collectively referred to as a body surface temperature measurement unit 16. The control unit 13 of the body surface temperature measurement unit 16 outputs temperature data to the specific site defining unit 21. The processing of the specific site defining unit 21, the temperature pattern calculation unit 22, and the subject distinguishing unit 24 is similar to the processing described in the first embodiment.

Note that the diseased person distinguishing device 2A includes a display unit 14A and an operation unit 15A.

The display unit 14A displays a thermal image on the basis of image data output from the control unit 13, similarly to the display unit 14 according to the first embodiment. The display unit 14A can also display the distinction result of the subject or the possibility that the subject is a diseased person, similarly to the display unit 25 according to the first embodiment.

The operation unit 15A inputs an instruction to cause the control unit 13 to output the absolute temperature of the measurement object, similarly to the operation unit 15 according to the first embodiment. The operation unit 15A can also cause the user using the diseased person distinguishing device 2A to check and input the content displayed on the display unit 14A, receive the distinction result as to whether the subject is a healthy person or a diseased person, and output the distinction result to the temperature pattern learning unit 27, similarly to the input unit 26 according to the first embodiment.

In the diseased person distinguishing device 2A according to the third embodiment described above, processing of the thermal image and determination of the subject can be performed by the single device. Therefore, the diseased person distinguishing device 2A is reduced in weight and size, and easy to carry.

### Fourth Embodiment

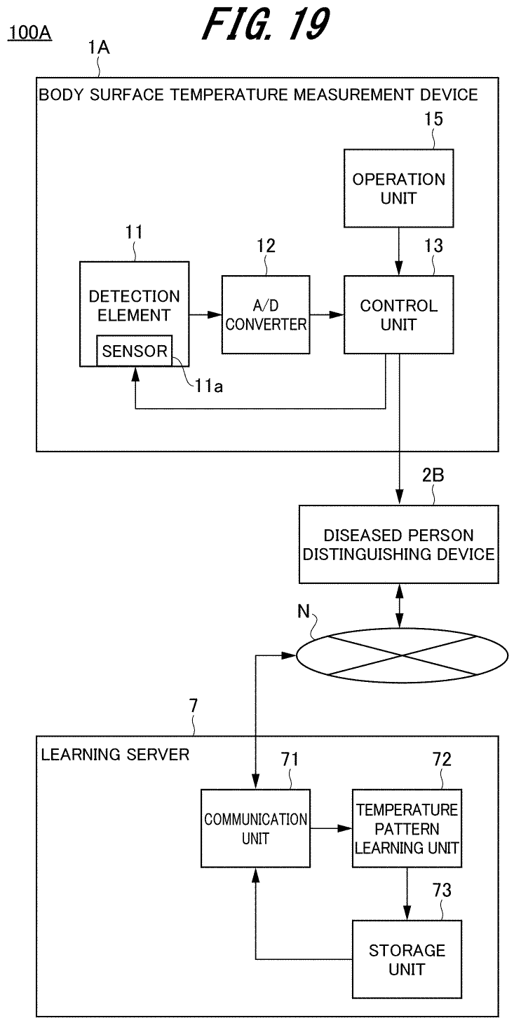

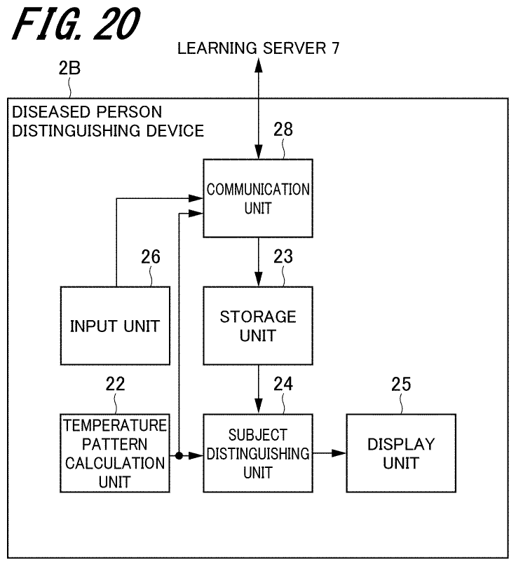

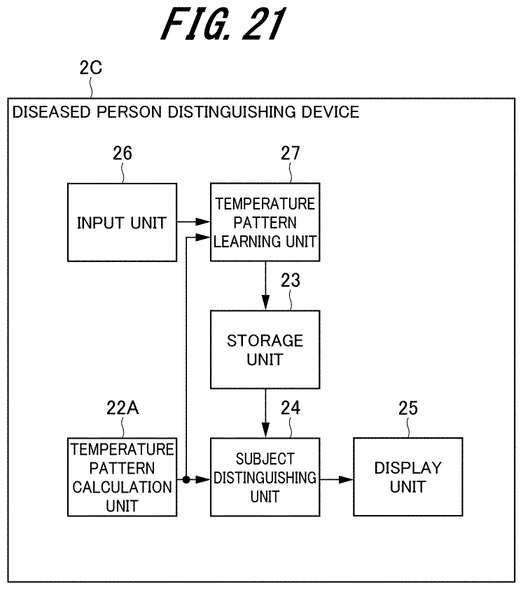

Next, a diseased person distinguishing system according to a fourth embodiment of the present invention will be described with reference to FIGS. 19 and 20.

The body surface temperature measurement device 1 including the display unit 14 capable of displaying a thermal image is very expensive. However, the technique for distinguishing between a diseased person and a healthy person using a temperature pattern may be used in various situations, and it is required to make the body surface temperature measurement device 1 inexpensive and easy to carry. Under such circumstances, a diseased person distinguishing system according to the present embodiment is configured to be able to distinguish between a diseased person and a healthy person even with the use of a non-contact body surface temperature measurement device without a display unit.

FIG. 19 is a block diagram illustrating an internal configuration example of a diseased person distinguishing system 100A.

The diseased person distinguishing system 100A includes a body surface temperature measurement device 1A, a diseased person distinguishing device 2B, and a learning server 7.

The body surface temperature measurement device 1A has a configuration in which the lens 10 and the display unit 14 are removed from the body surface temperature measurement device 1 illustrated in FIG. 1. The detection element 11 includes one sensor 11a. For example, the outer shape of the body surface temperature measurement device 1A is formed in a pen shape to enable the user to bring the body surface temperature measurement device 1A close to a specific site of the subject and measure the temperature of the specific site. It is assumed that the user, the body surface temperature measurement device 1A, the diseased person distinguishing device 2B, and the learning server 7 comprehend the measurement order of predetermined specific sites. Temperature data including the temperatures measured by the user at the specific sites of the subject using the body surface temperature measurement device 1A in a predetermined measurement order is output to the diseased person distinguishing device 2B in the measurement order. Note that the temperature data may be output every time the body surface temperature measurement device 1A measures each specific site, or may be output collectively after measuring all the specific sites for each subject.

Note that a liquid crystal display device or the like that displays the measured temperature of the specific site may be provided as a display unit in the body surface temperature measurement device 1A. The liquid crystal display device may display the name or the like of the specific site to be measured in the order of measurement. For example, the user inputs the fact that the temperature measurement of the specific site is finished by an operation such as pressing the operation unit 15 every time the temperature of the specific site is measured using the body surface temperature measurement device 1A. When the operation unit 15 is pressed, the name or the like of the specific site to be measured subsequently may be displayed on the liquid crystal display device.

The diseased person distinguishing device 2B can acquire the temperature data of the specific site from the body surface temperature measurement device 1A. The diseased person distinguishing device 2B can also transmit and receive data to and from the learning server 7 through a network N. The temperature data of she specific site acquired by the body surface temperature measurement device 1A and the user distinction result input by the user from the diseased person distinguishing device 2B are transmitted from the diseased person distinguishing device 2B to the learning server 7. Also, the diseased person distinguishing device 2B receives the data of a learned model from the learning server 7.

The learning server 7 includes a communication unit 71, a temperature pattern learning unit 72, and a storage unit 73. The learning server 7 can output, to the diseased person distinguishing device 2B, the temperature data of the specific site of the subject received from the diseased person distinguishing device 2B and the learned model learned using the user distinction result as an input.

The communication unit 71 can be connected to the diseased person distinguishing device 2B via the network N. The communication unit 71 receives the temperature data of the specific site of the subject and the user distinction result from the diseased person distinguishing device 2B. The communication unit 71 also transmits the learned model read from the storage unit 73 to the diseased person distinguishing device 2B.

The temperature pattern learning unit 72 generates and updates a learned model from a learning result obtained by learning a temperature pattern on the basis of the temperature data of the specific site received by the communication unit 71 from the diseased person distinguishing device 2B and the user distinction result input by the user. The learning processing performed by the temperature pattern learning unit 72 is similar to the learning processing performed by the temperature pattern learning unit 27 of the diseased person distinguishing device 2 according to the first embodiment. The temperature pattern learning unit 72 then stores the learned model in the storage unit 73.

The storage unit 73 stores the learned model learned by the temperature pattern learning unit 72. As described above, the learned model read from the storage unit 73 by the communication unit 71 is transmitted to the diseased person distinguishing device 2B via the network N.

FIG. 20 is a block diagram illustrating an internal configuration example of the diseased person distinguishing device 2B.

The diseased person distinguishing device 2B has a configuration in which the specific site defining unit 21 and the temperature pattern learning unit 27 are removed from the diseased person distinguishing device 2 illustrated in FIG. 3. The diseased person distinguishing device 2B includes a communication unit 28 capable of communicating with the learning server 7.

The temperature pattern calculation unit 22 calculates, as a temperature pattern, normalized temperature values for the specific sites measured in the predetermined measurement order by the user operating the body surface temperature measurement device 1A. The temperature pattern calculated by the temperature pattern calculation unit 22 is shaped for each subject.

The communication unit 28 transmits to the learning server 7 the temperature pattern calculated by the temperature pattern calculation unit 22 and the user distinction result input by the user through the input unit 26. The communication unit 28 also receives the learned model from the learning server 7 and stores the learned model in the storage unit 23.

The storage unit 23 stores the learned model received by the communication unit 28 from the learning server 7. The learned model is appropriately read from the storage unit 23 by the subject distinguishing unit 24 and used for determination of the subject.

The diseased person distinguishing system 100A according to the fourth embodiment described above includes the body surface temperature measurement device 1A capable of measuring the temperature for each specific site of the subject. The user brings the single body surface temperature measurement device 1A close to a plurality of specific sites of the subject in a specified measurement order to sequentially obtain the temperature of each specific site. Also, an appropriate learned model obtained by the learning server 7 performing machine learning of a temperature pattern is provided to the diseased person distinguishing device 2B. Therefore, the diseased person distinguishing device 2B can distinguish between a diseased person and a healthy person by applying the learned model received from the learning server 7 to the temperature pattern. In the present embodiment, the learning server 7 performs learning of a temperature pattern, which places a large load on the CPU 31 (refer to FIG. 4), to enable the specifications of the diseased person distinguishing device 2B to be lowered. Also, since the body surface temperature measurement device 1A does not include the display unit 14, the body surface temperature measurement device 1A can be downsized.

As described above, the diseased person distinguishing device 2B can distinguish between a diseased person and a healthy person by using the data of the body surface temperature measured for each specific site input from the body surface temperature measurement device 1A without using a thermal image as an input.

Also, the learning server 7 can learn temperature patterns corresponding to the body surface temperatures of the subjects measured in different regions by acquiring the temperature patterns and the user distinction results from the plurality of body surface temperature measurement devices 1A. For this reason, the accuracy of the temperature pattern is improved, and the accuracy of the distinction result between a diseased person and a healthy person derived using this temperature pattern is also improved.

### Fifth Embodiment

Next, a diseased person distinguishing device used in a diseased person distinguishing system according to a fifth embodiment of the present invention will be described with reference to FIG. 21. A diseased person distinguishing device according to the present embodiment derives as a temperature pattern the temperatures for the respective pixels of a thermal image input from the body surface temperature measurement device that outputs the thermal image, and identifies the subject.

FIG. 21 is a block diagram illustrating an internal configuration example of a diseased person distinguishing device 2C.

The diseased person distinguishing device 2C has a configuration in which the specific site defining unit 21 is removed from the diseased person distinguishing device 2 illustrated in FIG. 3. In addition, the diseased person distinguishing device 2C is connected to the body surface temperature measurement device 1 to constitute a diseased person distinguishing system according to the fifth embodiment.

The temperature data input from the body surface temperature measurement device 1 to a temperature pattern calculation unit 22A includes the entire thermal image obtained as the body surface temperature measurement device has photographed the subject. The temperature pattern calculation unit 22A has a function of locating face parts constituting the face on the basis of the thermal image of the face, similarly to the specific site defining unit 21 according to the first embodiment. Therefore, the temperature pattern calculation unit 22A locates face parts constituting the face on the basis of the temperature data of the entire thermal image and calculates, as a temperature pattern, temperature data extracted for the respective pixels of the thermal image. The temperature pattern calculated by the temperature pattern calculation unit 22A is output to the subject distinguishing unit 24 and the temperature pattern learning unit 27.

Since the operations of the storage unit 23, the subject distinguishing unit 24, the display unit 25, the input unit 26, and the temperature pattern learning unit 27 are similar to the operations of the respective units in the diseased person distinguishing device 2 illustrated in FIG. 3, detailed description thereof will be omitted.

In the diseased person distinguishing system according to the fifth embodiment described above, it is determined whether the subject is a healthy person or a diseased person on the basis of the temperature pattern calculated for the respective pixels of the thermal image input into the diseased person distinguishing device 2C. This can dispense with the processing for defining the specific site of the subject from the thermal image and reduce the volume of the processing from acquiring the thermal image to identifying the subject.

Also, in the diseased person distinguishing device 2C according to the fifth embodiment, the temperature pattern calculation unit 22A locates face parts constituting the face on the basis of the temperature data of the entire thermal image obtained by photographing the face and the neck of the subject. Furthermore, the temperature pattern calculation unit 22A may determine that the face of the subject faces the front in the same direction as the direction in which the body surface temperature measurement device 1 performs photographing, thereby calculating the temperature data extracted for the respective pixels of the thermal image as the temperature pattern. The temperature pattern calculated by the temperature pattern calculation unit 22A is output to the subject distinguishing unit 24 and the temperature pattern learning unit 27. The operations of the storage unit 23, the subject distinguishing unit 24, the display unit 25, the input unit 26, and the temperature pattern learning unit 27 in the diseased person distinguishing device 2C are similar to the operations of the respective units in the diseased person distinguishing device 2 illustrated in FIG. 3.

### MODIFICATION EXAMPLES

As the body surface temperature measurement device, a plurality of non-contact thermometers (radiation thermometers) may be used. Then, by simultaneously measuring a plurality of specific sites of the subject with the plurality of non-contact thermometers, the temperatures of the specific sites and the normalized temperature values may be obtained for each subject.

Also, in the body surface temperature measurement devices according to the third to fifth embodiments as well, the temperature of the neck of the subject, which is measured by the body surface temperature measurement device according to the second embodiment, may be included as the specific site. At this time, the temperature pattern learning unit 27 can perform machine learning and update the learned model on the basis of the temperature pattern of the neck and the specific site other than the neck of the subject. In addition, the subject distinguishing unit 24 can determine whether the subject is a healthy person or a diseased person by applying this learned model to the temperature pattern of the neck and the specific site other than the neck of the subject.

Also, it is to be understood that the present invention is not limited to each of the above-described embodiments but can take various other application examples and modification examples without departing from the spirit and scope of the present invention described in the claims.

For example, each of the above-described embodiments specifically describes the configurations of the device and the system in detail in order to facilitate understanding of the present invention and is not necessarily limited to one including the entire configuration described. Also, a part of the configuration of the embodiment described here can be replaced with the configuration of another embodiment, and furthermore, the configuration of another embodiment can be added to the configuration of a certain embodiment. Also, another configuration can be added to, deleted from, and replaced with a part of the configuration of each of the embodiments.

Also, control lines and information lines that are considered to be necessary for the description are illustrated, and not all of the control lines and information lines that are necessary as a product are illustrated. In practice, it may be considered that almost all of the components are connected to each other.

### REFERENCE SIGNS LIST

1 Body surface temperature measurement device
2 Diseased person distinguishing device
13 Control unit
14 Display unit
15 Operation unit
21 Specific site defining unit
22 Temperature pattern calculation unit
23 Storage unit
24 Subject distinguishing unit
25 Display unit
26 Input unit
27 Temperature pattern learning unit
40 Thermal image of face
50, 60 Temperature pattern
100 Diseased person distinguishing system

1. A diseased person distinguishing device comprising:
a temperature pattern calculation unit that calculates a temperature pattern from a temperature in an exposed region of a subject on a basis of temperature data input from a body surface temperature measurement device that measures a body surface temperature of the subject; and
a subject distinguishing unit that applies a learned model that has been learned in advance to the temperature pattern calculated to determine whether the subject is a diseased person or a healthy person.

2. The diseased person distinguishing device according to claim 1, further comprising:
a specific site defining unit that defines at least one specific site in the exposed region of the subject on the basis of the temperature data,
wherein the temperature pattern calculation unit calculates the temperature pattern expressed as a group of the temperature data collected from the plurality of specific sites.

3. The diseased person distinguishing device according to claim 2,
wherein at least one of the specific sites is any one of an eye inner corner, a nose tip, a nose side, a cheek, a jaw, an ear, a hand, a head excluding a hair portion, a temple, and a neck of the subject.

4. The diseased person distinguishing device according to claim 3,
wherein the temperature data includes a thermal image obtained as the body surface temperature measurement device photographs a face of the subject,
wherein the specific site defining unit locates face parts constituting the face on the thermal image and defines the specific sites on the thermal image, and
wherein the temperature pattern calculation unit extracts the temperature data from the specific sites defined and derives the temperature pattern of the plurality of specific sites.

5. The diseased person distinguishing device according to claim 3,
wherein the temperature data includes a thermal image obtained as the body surface temperature measurement device photographs a face of the subject, and
wherein the temperature pattern calculation unit locates face parts constituting the face and calculates, as the temperature pattern, the temperature data extracted for respective pixels of the thermal image.

6. The diseased person distinguishing device according to claim 3,
wherein the temperature data includes a thermal image obtained as the body surface temperature measurement device photographs a face and a neck of the subject,
wherein the specific site defining unit locates face parts constituting the face on the thermal image, and determines that the face of the subject faces front in an equal direction to a direction in which the body surface temperature measurement device performs photographing and defines the specific sites from the thermal image, and
wherein the temperature pattern calculation unit extracts the temperature data from the specific sites defined and derives the temperature pattern of the plurality of specific sites.

7. The diseased person distinguishing device according to claim 3,
wherein the temperature data includes a thermal image obtained as the body surface temperature measurement device photographs a face and a neck of the subject, and
wherein the temperature pattern calculation unit locates face parts constituting the face on the thermal image, and determines that the face of the subject faces front in an equal direction to a direction in which the body surface temperature measurement device performs photographing and calculates, as the temperature pattern, the temperature data extracted for respective pixels of the thermal image.

8. The diseased person distinguishing device according to claim 2, further comprising:
a storage unit that stores the learned model,
wherein the subject distinguishing unit identifies the subject on a basis of the learned model read from the storage unit.

9. The diseased person distinguishing device according to claim 8, further comprising:
a display unit,
wherein the subject distinguishing unit displays on the display unit a distinction result by the subject distinguishing unit or possibility that the subject is the diseased person.

10. The diseased person distinguishing device according to claim 9, further comprising:
an input unit that receives a user distinction result as to whether the subject is a healthy person or a diseased person; and
a temperature pattern learning unit that generates the learned model learned by means of a machine learning model on a basis of the user distinction result and the temperature pattern used in the subject distinguishing unit.

11. The diseased person distinguishing device according to claim 1,
wherein the temperature data includes temperatures that the body surface temperature measurement device has measured at specific sites of the subject in a predetermined measurement order, and
wherein the temperature pattern calculation unit calculates, as the temperature pattern, the temperatures for the specific sites measured in the measurement order.

12. A diseased person distinguishing system comprising:
a diseased person distinguishing device; and
a learning server,
wherein the diseased person distinguishing device includes
a temperature pattern calculation unit that calculates a temperature pattern from a temperature in an exposed region of a subject on a basis of temperature data input from a body surface temperature measurement device that measures a body surface temperature of the subject, and
a subject distinguishing unit that applies a learned model that has been learned in advance to the temperature pattern calculated to determine whether the subject is a diseased person or a healthy person, and
wherein the learning server includes
a temperature pattern learning unit that learns the temperature pattern by means of a machine learning model and generates the learned model.

13. The diseased person distinguishing system according to claim 12,
wherein the diseased person distinguishing device includes an input unit that receives a user distinction result as to whether the subject is a healthy person or a diseased person, and
wherein the temperature pattern learning unit generates the learned model on a basis of the user distinction result and the temperature pattern used in the subject distinguishing unit.
